(19) United States
(12) Patent Application Publication
(10) Pub. No.: US 2021/0275272 A1
(43) Pub. Date: Sep. 9, 2021

(54) Implantable Force Gauges

(71) Applicant: 4Tech Inc., Waltham, MA (US)

(72) Inventors: Gabriel Sobrino-Serrano, Kinvara (IE); Idan Tobis, Beth Hashmonai (IL)

(73) Assignee: 4Tech Inc., Waltham, MA (US)

(21) Appl. No.: 16/484,404

(22) PCT Filed: Feb. 7, 2018
(86) PCT No.: PCT/US2018/017284

§ 371 Date: Aug. 7, 2019

Related U.S. Application Data
(60) Provisional application No. 62/456,206, filed on Feb. 8, 2017.

(51) Int. Cl. A61B 90/00; A61F 2/24; A61B 17/04

(52) U.S. Cl. CPC A61B 90/06; A61F 2/2466; A61B 90/39; A61B 17/0401; A61B 2017/0464; A61B 2090/064; A61B 2090/0807; A61B 2090/3966; A61F 2/2454

(57) ABSTRACT

A tension system (10) is provided, including first and second tissue anchors (20A, 20B) configured to be anchored to two target sites, respectively; and first and second tethers (24A, 24B), coupled to the first and the second tissue anchors (20A, 20B), respectively. An implantable force gauge (30) includes first and second components (31A, 31B), which are fixed to the first and the second tethers (24A, 24B), respectively, and which are non-integral with each other and are configured to be coupled together in situ so as to couple the first and the second tissue anchors (20A, 20B) together via the first and the second tethers (24A, 24B), for applying variable tension between the two target sites. The implantable force gauge (30) is configured to provide a radiographically-discernible indication of a magnitude of the variable tension between the two target sites, enabling radiographically monitoring of changes in the magnitude of the tension

### CROSS-REFERENCE TO RELATED APPLICATIONS

This application is a U.S. national stage application filed pursuant to 35 U.S.C. § 371 from International Patent Application PCT/US2018/017284, filed on Feb. 7, 2018, which claims the benefit of priority and the filing date of U.S. Provisional Application 62/456,206, filed Feb. 8, 2017, which is assigned to the assignee of the present application and each of which is incorporated herein by reference.

### FIELD OF THE APPLICATION

The present invention relates generally to minimally-invasive valve repair, and more specifically to minimally-invasive methods for repairing cardiac valves.

### BACKGROUND OF THE APPLICATION

Functional tricuspid regurgitation (FTR) is governed by several pathophysiologic abnormalities such as tricuspid valve annular dilatation, annular shape, pulmonary hypertension, left or right ventricle dysfunction, right ventricle geometry, and leaflet tethering. Treatment options for FTR are primarily surgical.

U.S. Pat. No. 8,475,525 to Maisano et al. describes a method that includes implanting at least a first tissue-engaging element in a first portion of tissue in a vicinity of a heart valve of a patient, implanting at least a second tissue-engaging element in a portion of a blood vessel that is in contact with an atrium of a heart of the patient, and drawing at least a first leaflet of the valve toward at least a second leaflet of the valve by adjusting a distance between the portion of the blood vessel and the first portion of tissue in the vicinity of the heart valve of the patient. In one configuration, a proximal end portion of a longitudinal member is shaped so as to define one or more engaging elements (e.g., hooks or barbs), which are coupleable with the struts of a stent member in order to maintain the tension applied to a longitudinal member for remodeling the tricuspid valve.

U.S. Pat. No. 6,045,497 to Schweich, Jr. et al. describes an apparatus for treatment of a failing heart by reducing the wall tension therein. The apparatus may include a tension member for drawing at least two walls of a heart chamber toward each other. The tension member may be radiopaque, echo-cardiographic compatible, or MRI-compatible or includes a marker which is radiopaque, echo-compatible, or MRI-compatible. Providing radiopaque echo-compatible or MRI-compatible tension members or markers may allow follow-up, non-invasive monitoring of the tension member after implantation. The presence of the tension member can be visualized and the distance between two or more markers measured. In addition, a strain gauge may be disposed on the tension member to monitor the loading on the member in use.

### SUMMARY OF THE APPLICATION

Some applications of the present invention provide an implantable force gauge, which is configured to provide a radiographically-discernible indication of a magnitude of variable tension applied between two target tissue sites, thereby enabling (a) radiographically ascertaining, from outside the patient's body, of the magnitude of the variable tension during the applying of the variable tension, and (b) radiographically monitoring, from outside the patient's body, of changes in the magnitude of the variable tension over time after implantation of the tension system. The implantable force gauge thus provides a simple, non-invasive way to monitor variable tension both during the implantation procedure and over time after the procedure, without the need for more complex and invasive measurement techniques.

There is therefore provided, in accordance with an application of the present invention, a tension system for applying variable tension between two target sites in a patient's body, the tension system including:

first and second tissue anchors, including respective first and second tissue-coupling elements that are configured to be anchored to the two target sites, respectively;

first and second tethers, coupled to the first and the second tissue anchors, respectively; and

an implantable force gauge, which includes first and second components, which are fixed to the first and the second tethers, respectively, and which are non-integral with each other and are configured to be coupled together in situ so as to couple the first and the second tissue anchors together via the first and the second tethers, for applying the variable tension between the two target sites, the implantable force gauge configured to provide a radiographically-discernible indication of a magnitude of the variable tension between the two target sites, thereby enabling (a) radiographically ascertaining, from outside the patient's body, of the magnitude of the variable tension during the applying of the variable tension, and (b) radiographically monitoring, from outside the patient's body, of changes in the magnitude of the variable tension over time after implantation of the tension system.

For some applications, the first component of the implantable force gauge includes a longitudinally deformable element, which optionally includes a spring or is longitudinally plastically deformable.

For some applications, the implantable force gauge includes a plurality of radiographically-discernible fiducial markers and a radiographically-discernible pointer, the implantable force gauge is arranged such that the pointer moves longitudinally with respect to the fiducial markers so as to provide the radiographically-discernible indication of the magnitude of the variable tension, and the pointer longitudinally coincides with different ones of the fiducial markers at different respective values of the magnitude of the variable tension. Optionally, the pointer has a radiopacity different from a radiopacity of the fiducial markers.

There is further provided, in accordance with an application of the present invention, a tension system for applying variable tension between first and second target sites in a patient's body, the tension system including:

(a) a first tissue anchor, which includes:
an anchor shaft;
a first tissue-coupling element, which (i) extends from a distal end of the anchor shaft, and (ii) includes a wire, which is shaped as an open shape when the first tissue anchor is unconstrained by a deployment tool, and which is configured to be anchored to the first target site; and
a flexible elongate tension member, which includes (i) a distal portion that is fixed to a site on the open shape, (ii) a proximal portion, at least a portion of which runs alongside at least a portion of the anchor shaft, and (iii) a crossing portion, which crosses from the site on the open shape to the distal end of the anchor shaft when the first tissue anchor is unconstrained by the deployment tool, wherein the first tissue anchor is configured to allow relative axial motion between the at least a portion of the anchor shaft and the at least a portion of the proximal portion of the flexible elongate tension member when the first tissue anchor is unconstrained by the deployment tool;

(b) a second tissue anchor, which includes a second tissue-coupling element that is configured to be anchored to the second target site;

(c) one or more tethers, configured to couple the flexible elongate tension member to the second tissue anchor and apply the variable tension between the first and the second target sites; and

(d) an implantable force gauge, which includes:
one or more radiographically-discernible fiducial markers, which are disposed on the proximal portion of the flexible elongate tension member; and
a radiographically-discernible pointer, which is disposed on the anchor shaft,
wherein the implantable force gauge is arranged such that the one or more fiducial markers move longitudinally with respect to the pointer so as to provide a radiographically-discernible indication of a magnitude of the variable tension between the first and the second target sites, thereby enabling (a) radiographically ascertaining, from outside the patient's body, of the magnitude of the variable tension during the applying of the variable tension, and (b) radiographically monitoring, from outside the patient's body, of changes in the magnitude of the variable tension over time after implantation of the tension system, wherein the pointer longitudinally coincides with different ones of the fiducial markers at different respective values of the magnitude of the variable tension.

There is still further provided, in accordance with an application of the present invention, a method for applying variable tension between two target sites in a patient's body, the method including:

anchoring, to the two target sites, respectively, first and second tissue-coupling elements of respective first and second tissue anchors of a tension system;

coupling the first and the second tissue anchors together in situ via first and second tethers of the tension system, coupled to the first and the second tissue anchors, respectively, by coupling together in situ first and second components of an implantable force gauge of the tension system, the first and the second components are non-integral with each other and are fixed to the first and the second tethers, respectively, and the implantable force gauge is configured to provide a radiographically-discernible indication of a magnitude of the variable tension between the two target sites;

applying the variable tension between the two target sites via the first and the second tethers and the implantable force gauge; and

radiographically ascertaining, from outside the patient's body, the magnitude of the variable tension during the applying of the variable tension, by radiographically observing the radiographically-discernible indication of the magnitude of the variable tension.

For some applications, the method further includes radiographically monitoring, from outside the patient's body, changes in the magnitude of the variable tension over time after implantation of the tension system.

For some applications, the first component of the implantable force gauge includes a longitudinally deformable element, which optionally includes a spring or is longitudinally plastically deformable.

For some applications, the implantable force gauge includes a plurality of radiographically-discernible fiducial markers and a radiographically-discernible pointer, the implantable force gauge is arranged such that the pointer moves longitudinally with respect to the fiducial markers so as to provide the radiographically-discernible indication of the magnitude of the variable tension, and the pointer longitudinally coincides with different ones of the fiducial markers at different respective values of the magnitude of the variable tension. Optionally, the pointer has a radiopacity different from a radiopacity of the fiducial markers.

For some applications, the two target sites are two cardiac tissue target sites, respectively.

There is additionally provided, in accordance with an application of the present invention, a method for applying variable tension between first and second target sites in a patient's body, the method including:

anchoring, to the first target site, a wire of a first tissue-coupling element of a first tissue anchor of a tension system, wherein the first tissue anchor further includes an anchor shaft, wherein the first tissue-coupling element extends from a distal end of the anchor shaft, wherein the wire is shaped as an open shape when the first tissue anchor is unconstrained by a deployment tool, and wherein the first tissue anchor further includes a flexible elongate tension member, which includes (i) a distal portion that is fixed to a site on the open shape, (ii) a proximal portion, at least a portion of which runs alongside at least a portion of the anchor shaft, and (iii) a crossing portion, which crosses from the site on the open shape to the distal end of the anchor shaft when the first tissue anchor is unconstrained by the deployment tool, wherein the first tissue anchor is configured to allow relative axial motion between the at least a portion of the anchor shaft and the at least a portion of the proximal portion of the flexible elongate tension member when the first tissue anchor is unconstrained by the deployment tool;

anchoring, to the second target site, a second tissue-coupling element of a second tissue anchor of the tension system, such that one or more tethers couple the flexible elongate tension member to the second tissue anchor and apply the variable tension between the first and the second target sites; and

radiographically ascertaining, from outside the patient's body, the magnitude of the variable tension during the applying of the variable tension, by radiographically observing a radiographically-discernible indication of the magnitude of the variable tension provided by an implantable force gauge of the tension system, which includes (a) one or more radiographically-discernible fiducial markers, which are disposed on the proximal portion of the flexible elongate tension member, and (b) a radiographically-discernible pointer, which is disposed on the anchor shaft, wherein the implantable force gauge is arranged such that the one or more fiducial markers move longitudinally with respect to the pointer so as to provide the radiographically-discernible indication of the magnitude of the variable tension between the first and the second target sites, and the pointer longitudinally coincides with different ones of the fiducial markers at different respective values of the magnitude of the variable tension.

For some applications, the method further includes radiographically ascertaining, from outside the patient's body, changes in the magnitude of the variable tension over time after implantation of the tension system.

For some applications, the first and the second target sites are two cardiac tissue target sites, respectively.

The present invention will be more fully understood from the following detailed description of embodiments thereof, taken together with the drawings, in which:

### BRIEF DESCRIPTION OF THE DRAWINGS

FIG. 1 is a schematic illustration of a tension system for applying variable tension between two target sites in a patient's body, in accordance with an application of the present invention;

FIGS. 2A-C are schematic illustrations of a method for applying variable tension between two target sites in a patient's body using the tension system of FIG. 1, in accordance with an application of the present invention;

FIGS. 3A-E are schematic illustrations of another tension system for applying variable tension between two target sites in a patient's body, and a method for applying variable tension using the tension system, in accordance with respective applications of the present invention; and

FIGS. 4A-B are schematic illustrations of respective configurations of yet another tension system for applying variable tension between two target sites in a patient's body, in accordance with respective applications of the present invention.

### DETAILED DESCRIPTION OF APPLICATIONS

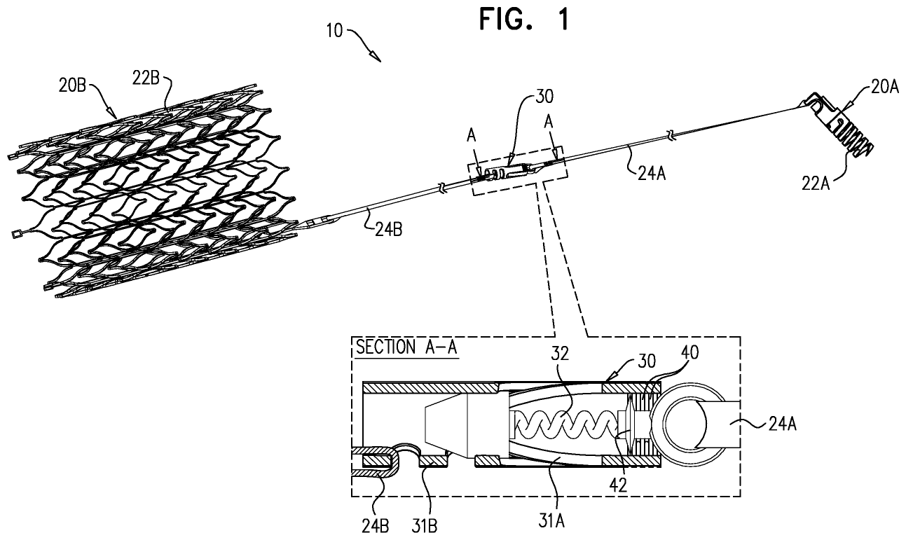

FIG. 1 is a schematic illustration of a tension system 10 for applying variable tension between two target sites in a patient's body, in accordance with an application of the present invention. Tension system 10 comprises first and second tissue anchors 20A and 20B, which comprise respective first and second tissue-coupling elements 22A and 22B that are configured to be anchored to the two target sites, respectively. Tension system 10 further comprises first and second tethers 24A and 24B, which are coupled to first and second tissue anchors 20A and 20B, respectively.

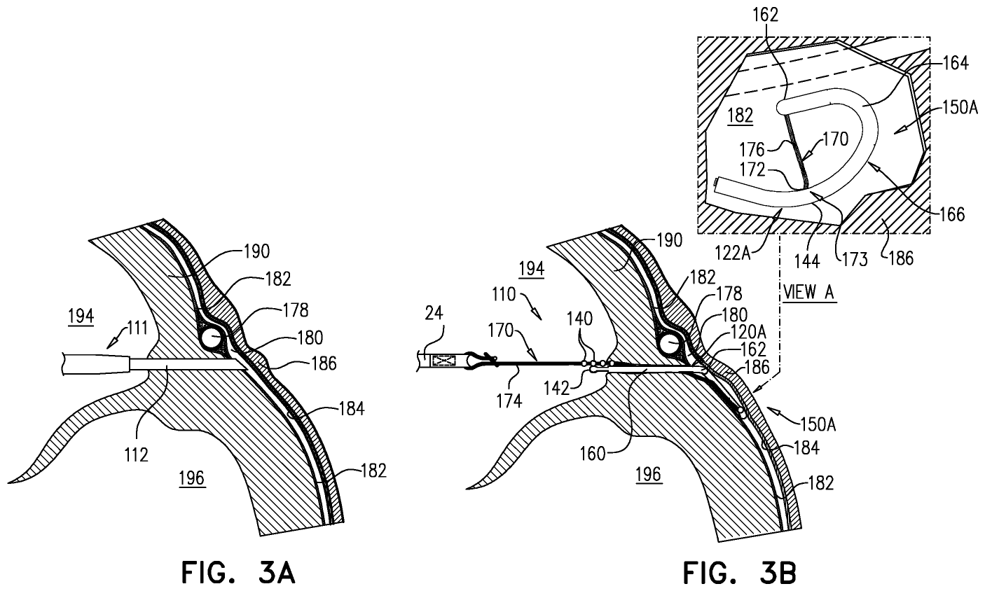

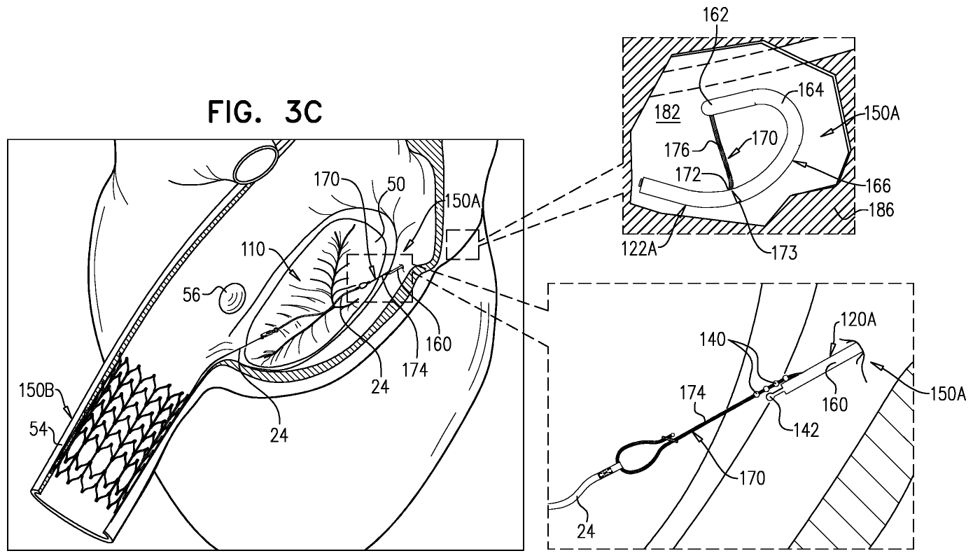

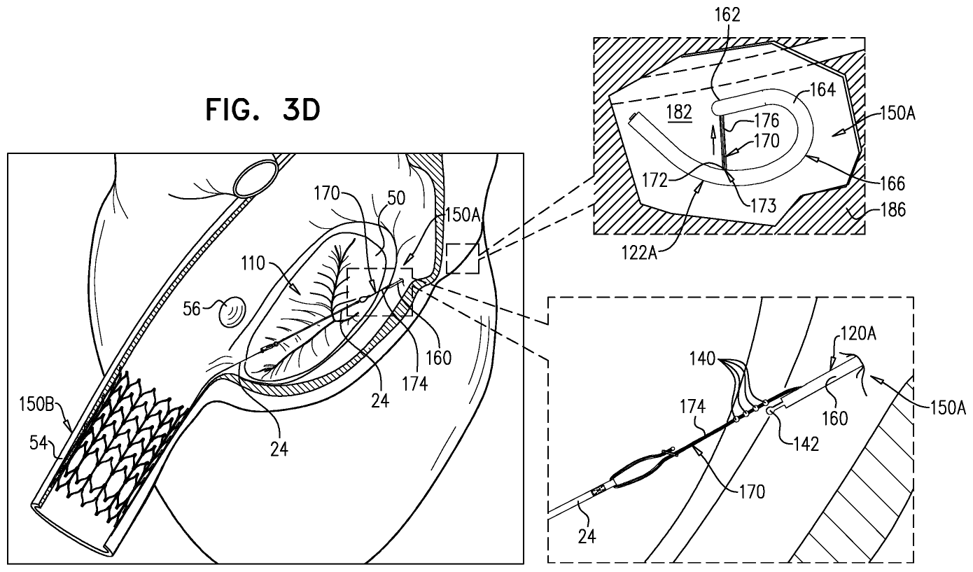

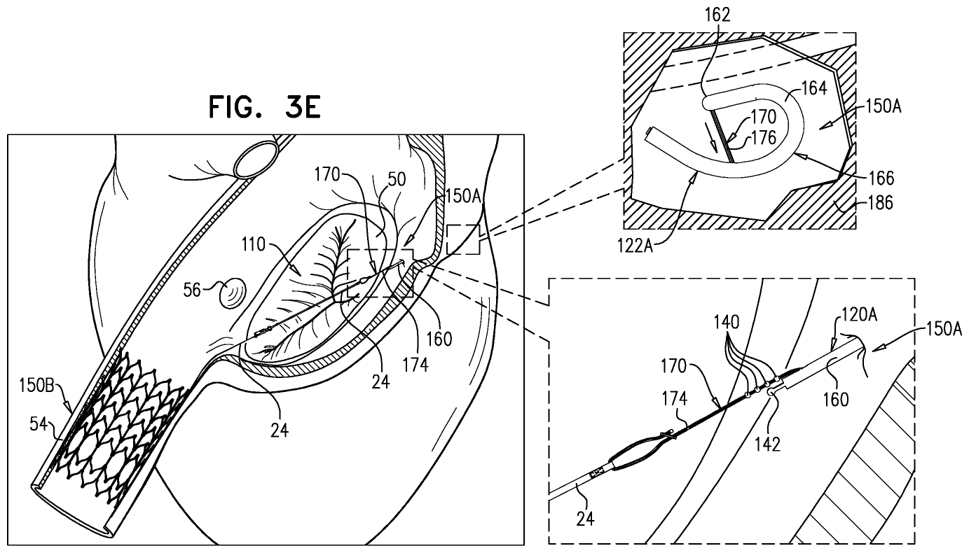

For some applications, first tissue-coupling element 22A is helical, as shown in FIG. 1; for other applications, first tissue-coupling element 22A comprises first tissue-coupling element 122A, described hereinbelow with reference to FIGS. 3A-E, or another tissue-coupling element. Alternatively or additionally, for some applications, as shown in FIG. 1, second tissue-coupling element 22B comprises a stent that comprises a plurality of struts. For some applications, fibrous glue is applied to one or both of the tissue-coupling elements to help secure the anchor in place and minimize detachment. Optionally, tissue-growth-enhancing coating is also applied to one or both of the tissue-coupling elements.

Tension system 10 further comprises an implantable force gauge 30, which comprises first and second components 31A and 31B, which are fixed to first and second tethers 24A and 24B, respectively. First and second components 31A and 31B are non-integral with each other and are configured to be coupled together in situ so as to couple the first and the second tissue anchors together via first and second tethers 24A and 24B, thereby applying the variable tension between the two target sites. Typically, implantable force gauge 30 is mechanical and does not operate using electricity or any non-mechanical energy.

Implantable force gauge 30 is configured to provide a radiographically-discernible indication of a magnitude of the variable tension between the two target sites, thereby enabling (a) radiographically ascertaining, from outside the patient's body, of the magnitude of the variable tension during implantation of tension system 10, such as described hereinbelow with reference to FIG. 1B, and (b) radiographically monitoring, from outside the patient's body, of changes in the magnitude of the variable tension over time after the implantation of tension system 10, such as described hereinbelow with reference to FIG. 1C.

For some applications, first component 31A of implantable force gauge 30 comprises a longitudinally deformable element 32. For some applications, longitudinally deformable element 32 comprises a spring, which is configured to elastically deform upon application of up to a certain force. For other applications, longitudinally deformable element 32 is longitudinally plastically deformable, upon application of beyond a certain force, in which case implantable force gauge 30 measures greatest force ever applied between the between the two target sites.

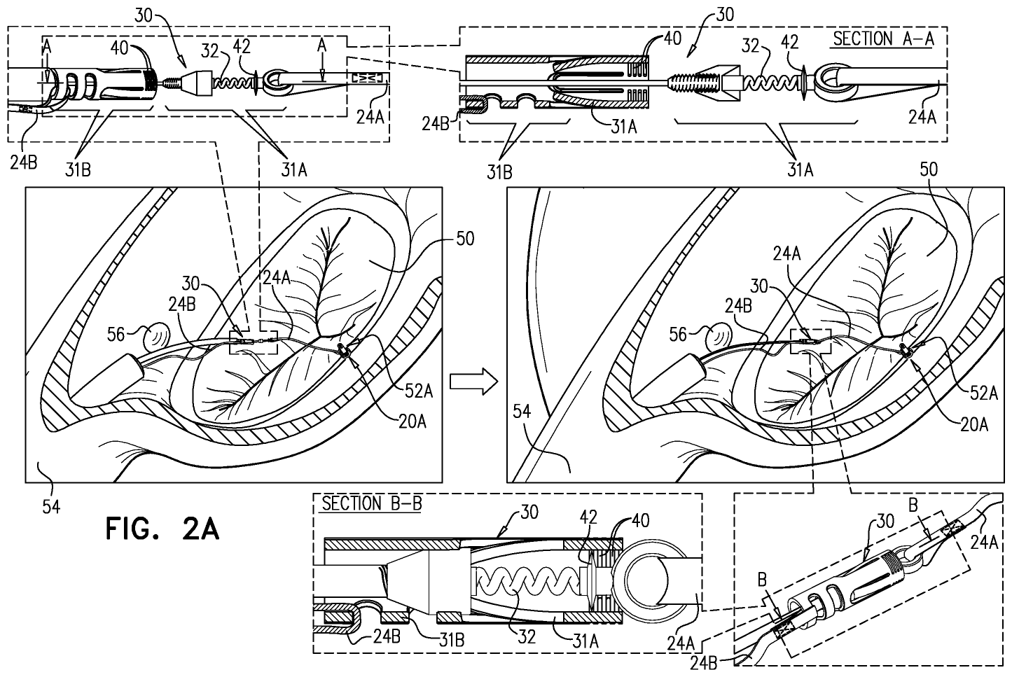

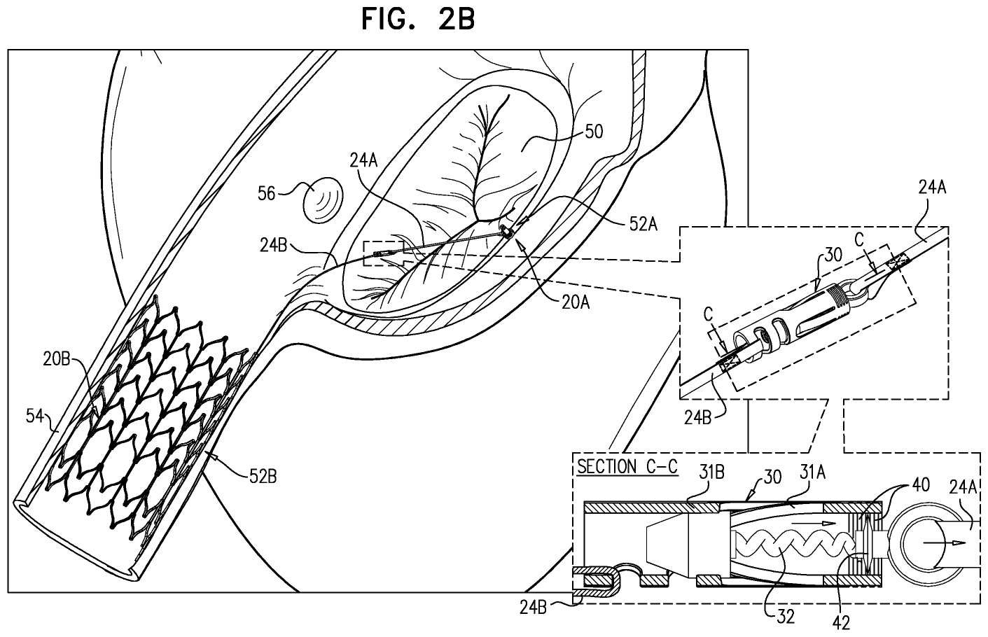

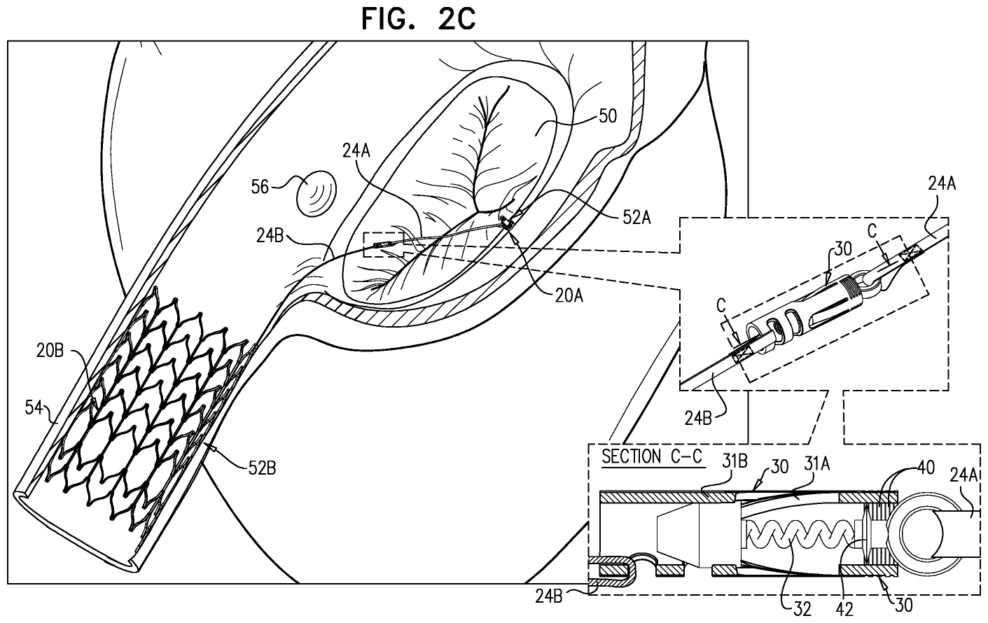

For some applications, implantable force gauge 30 comprises a plurality of radiographically-discernible fiducial markers 40 and a radiographically-discernible pointer 42, which, for example, may be shaped as a disc to enable clear imaging from many directions. Implantable force gauge 30 is arranged such that pointer 42 moves longitudinally with respect to fiducial markers 40 so as to provide the radiographically-discernible indication of the magnitude of the variable tension. Pointer 42 longitudinally coincides with different ones of fiducial markers 40 at different respective values of the magnitude of the variable tension, as described hereinbelow with reference to FIGS. 2A-C. Optionally, pointer 42 has a radiopacity different from a radiopacity of fiducial markers 40, to enhance visibility in the radiographic images.

Reference is now made to FIGS. 2A-C, which are schematic illustrations of a method for applying variable tension between two target sites in a patient's body using tension system 10, in accordance with an application of the present invention. For some applications, the two target sites are two cardiac tissue target sites, respectively, and tension system 10 is used for treating a heart of a patient. For some applications, such as shown, tension system 10 is used to treat a tricuspid valve 50, such as by reducing tricuspid valve regurgitation.

As shown in FIGS. 2A and 2B, first and second tissue-coupling elements 22A and 22B of respective first and second tissue anchors 20A and 20B are anchored to two target sites 50A and 50B, respectively (first tissue anchor 20A may be anchored before or after second tissue anchor 20B is anchored). For example, first tissue anchor 20A may be implanted in cardiac tissue of the patient, such as in the vicinity of tricuspid valve 50, and second tissue anchor 20B may be implanted in the patient, either before or after implanting first tissue anchor 20A, such as in a superior vena cava (SVC), an inferior vena cava (IVC) 54 (as shown), or a coronary sinus 56. Typically, first and second tissue anchors 20A and 20B are implanted in a transcatheter procedure (typically endovascularly, such as percutaneously), via a catheter, such as described in the applications incorporated hereinbelow by reference. During the implantation procedure, first and second tissue anchors 20A and 20B are coupled together in situ via first and second tethers 24A and 24B, by coupling together in situ first and second components 31A and 31B of implantable force gauge 30, such as using techniques described in one or more of the applications incorporated by reference hereinbelow (for example, using techniques described with reference to FIGS. 26-26, 29-30D, 31 and/or 32 in US Patent 9,241,702 to Maisano et al., and/or with reference to FIGS. 33A-B, 34A-E, 35A-C, 36A-B, 37A-B, 38A-C, 39A-B, and/or 40A-E in US Patent 9,307,980 to Gilmore et al.). First tissue anchor 20A may be anchored to first target site 50A before or after first and second components 31A and 31B of implantable force gauge 30 are coupled together in situ, and second tissue anchor 20B may be anchored to second target site 50B before or after first and second components 31A and 31B of implantable force gauge 30 are coupled together in situ.

As shown in FIG. 2A, pointer 42 longitudinally coincides with a first one of fiducial markers 40 at the relatively low tension (e.g., no tension) at this stage of the implantation procedure. At this stage of the implantation procedure, tethers 24A and 24B are typically slack, i.e., do not apply tension between first and second tissue anchors 20A and 20B. The relative location of pointer 42 with respect to fiducial markers 40 provides the radiographically-discernible indication of the magnitude of the variable tension.

As shown in FIG. 2B, the variable tension is applied between first and second target sites 50A and 50B via first and second tethers 24A and 24B and implantable force gauge 30. Upon application of the tension, pointer 42 longitudinally moves with respect to fiducial markers 40 (to the right in the figures), until pointer 42 longitudinally coincides with a second one of fiducial markers 40 at a relatively greater tension than the tension at the stage of the implantation procedure shown in FIG. 2A. The magnitude of the variable tension during implantation of the first and the second tissue anchors is radiographically ascertained, from outside the patient's body, by radiographically observing (e.g., by fluoroscopy or X-ray) the relative location of pointer 42 with respect to fiducial markers 40, i.e., the radiographically-discernible indication of the magnitude of the variable tension. Typically, during the implantation procedure, the magnitude of the tension is adjusted, optionally repeatedly, based on the radiographically-discernible indication of the magnitude of the tension, until a desire magnitude of tension is achieved.

For some applications, as shown in FIG. 2C, after completion of the implantation procedure (e.g., at least 24 hours, such as at least one week, one month, or at least one year after completion of the implantation procedure), changes in the magnitude of the variable tension are radiographically monitoring, from outside the patient's body, over time after the implantation of tension system 10. (Completion of the implantation procedure may be measured from the application of the variable tension between the first and the second target sites 50A and 50B during the implantation procedure.) For example, in FIG. 2C, tension system 10 is shown as having ceased to apply any tension between first and second tissue anchors 20A and 20B (first and second tethers 24A and 24B are slack). Alternatively, tension system 10 may still apply tension, but less than initially upon completion of the implantation procedure, such as shown in FIG. 3E for tension system 110. A physician may evaluate the continued efficacy of tension system 10 based on the radiographically-monitored tension, without the need to perform an invasive and/or more complex diagnostic procedure, and may decide to perform a follow-up adjustment procedure if necessary.

Reference is now made to FIGS. 3A-E, which are schematic illustrations of a tension system 110 for applying variable tension between two target sites in a patient's body, in accordance with an application of the present invention. Tension system 110 typically comprises first and second tissue anchors 120A and 120B, which comprise respective first and second tissue-coupling elements 122A and 122B that are configured to be anchored to first and second target sites 150A and 150B, respectively.

First tissue anchor 120A typically further comprises an anchor shaft 160. First tissue-coupling element 122A typically (i) extends from a distal end 162 of anchor shaft 160, and (ii) comprises a wire 164, which is shaped as an open shape 166, e.g., an open coil shape, that is typically generally orthogonal to anchor shaft 160 when first tissue anchor 120A is unconstrained by a deployment tool 111.

For some applications, first tissue anchor 120A further comprises a flexible elongate tension member 170, which includes:
a distal portion 172 that is fixed to a site 173 on open shape 166,
a proximal portion 174, at least a portion of which runs alongside at least a portion of anchor shaft 160 (e.g., inside at least a portion of anchor shaft 160, as shown), and
a crossing portion 176, which crosses from site 173 on open shape 166 to distal end 162 of anchor shaft 160 when first tissue anchor 120A is unconstrained by deployment tool 111.
   First tissue anchor 120A is configured to allow relative axial motion between the at least a portion of anchor shaft 160 and the at least a portion of proximal portion 174 of flexible elongate tension member 170 when first tissue anchor 120A is unconstrained by deployment tool 111.

For some applications, first tissue anchor 120A implements one or more of the features of tissue anchors described in PCT Publication WO 2016/087934, such as with reference to FIGS. 5A-D, 6A-B, 7A-B, 8A-B, and/or 9A-I thereof, and/or in US Patent Application Publication 2016/0262741, such as with reference to FIGS. 1A-D, 2A-B, 3A-D, 4A-B, 7, 8, 9A-F, 10A-B, 10H, and/or 13A-E.

Tension system 110 further comprises one or more tethers 24, which are configured to couple flexible elongate tension member 170 to second tissue anchor 120B and apply the variable tension between first and second target sites 150A and 150B.

Tension system 110 still further comprises an implantable force gauge 130, which comprises (a) one or more radiographically-discernible fiducial markers 140, which are disposed on proximal portion 174 of flexible elongate tension member 170, and (b) a radiographically-discernible pointer 142, which is disposed on anchor shaft 160.

Implantable force gauge 130 is arranged such that the one or more fiducial markers 140 move longitudinally with respect to pointer 142 so as to provide a radiographically-discernible indication of a magnitude of the variable tension between first and second target sites 150A and 150B, thereby enabling (a) radiographically ascertaining, from outside the patient's body, of the magnitude of the variable tension during the applying of the variable tension, such as described hereinbelow with reference to FIG. 3C-D, and (b) radiographically monitoring, from outside the patient's body, of changes in the magnitude of the variable tension over time after the implantation of tension system 110, such as described hereinbelow with reference to FIG. 3E. Pointer 142 longitudinally coincides with different ones of the fiducial markers 140 at different respective values of the magnitude of the variable tension. Typically, implantable force gauge 130 is mechanical and does not operate using electricity or any non-mechanical energy. For some applications, the one or more fiducial markers 140 comprise a plurality of fiducial markers 140, such as shown, e.g., at least three fiducial markers 140. For some applications, the one or more fiducial markers 140 are shaped as beads.

Reference is still made to FIGS. 3A-E, which additionally illustrate a method for applying variable tension between two target sites in a patient's body using tension system 110, in accordance with an application of the present invention. For some applications, the two target sites are two cardiac tissue target sites, respectively, and tension system 110 is used for treating a heart of a patient. For some applications, such as shown, tension system 110 is used to treat tricuspid valve 50, such as by reducing tricuspid valve regurgitation. Typically, first and second tissue anchors 120A and 120B are implanted in a transcatheter procedure (typically endovascularly, such as percutaneously), via a catheter, such as described in the applications incorporated hereinbelow by reference.

As shown in FIGS. 3A-B, wire 164 of first tissue-coupling element 122A of first tissue anchor 120A is anchored to first target site 150A. For some applications, as shown in FIG. 3A, first tissue-coupling element 122A is delivered to first target site 150A in an unexpanded generally elongate configuration within deployment tool 111, which comprises a hollow needle 112. The cardiac chamber may be a right atrium 194 (as shown), a right ventricle 196 (configuration not shown), a left atrium (configuration not shown), or a left ventricle (configuration not shown). In one application, hollow needle 112 is used to puncture through a first side of a myocardial tissue wall 190 and visceral pericardium 182 (which is part of the epicardium), avoiding vasculature such as the right coronary artery (RCA) 178. Hollow needle 112 is then further directed into a pericardial cavity 180 between visceral pericardium 182 and parietal pericardium 184, carefully avoiding puncturing parietal pericardium 184 and fibrous pericardium 186.

For some applications, as shown in FIG. 3B, first tissue-coupling element 122A is delivered through myocardial tissue wall 190 and into pericardial cavity 180, generally alongside and against the pericardial tissue. First tissue-coupling element 122A expands to the open shape on the second side of myocardial tissue wall 190, thereby anchoring first tissue-coupling element 122A to myocardial tissue wall 190.

Alternatively, first tissue-coupling element 122A is advanced within myocardial tissue wall 190, or is otherwise anchored to cardiac tissue at first target site 150A.

As shown in FIG. 3C, second tissue-coupling element 122B of second tissue anchor 120B is anchored to second target site 150B, such that one or more tethers 24 couple flexible elongate tension member 170 to second tissue anchor 120B and apply variable tension between first and second target sites 150A and 150B.

Also as shown in FIG. 3C, pointer 142 longitudinally coincides with a first one of fiducial markers 140 at the relatively low tension (e.g., no tension) at this stage of the implantation procedure. At this stage of the implantation procedure, the one or more tethers 24 are typically slack, i.e., do not apply tension between first and second tissue anchors 120A and 120B. The relative location of pointer 142 with respect to fiducial markers 140 provides the radiographically-discernible indication of the magnitude of the tension.

As shown in FIG. 3D, the variable tension is applied between first and second target sites 150A and 150B via the one or more tethers 24. Upon application of the variable tension, fiducial markers 140 longitudinally move with respect to pointer 142 (to the left in the figures), until pointer 142 longitudinally coincides with a second one of fiducial markers 140 at a relatively greater tension than the tension at the stage of the implantation procedure shown in FIG. 3C. The magnitude of the tension during implantation of the first and the second tissue anchors is radiographically ascertained, from outside the patient's body, by radiographically observing (e.g., by fluoroscopy or X-ray) the relative location of pointer 142 with respect to fiducial markers 140, i.e., the radiographically-discernible indication of the magnitude of the variable tension. Typically, during the implantation procedure, the magnitude of the tension is adjusted, optionally repeatedly, based on the radiographically-discernible indication of the magnitude of the tension, until a desire magnitude of tension is achieved.

For some applications, as shown in FIG. 3E, after completion of the implantation procedure (e.g., at least 24 hours, such as at least one week, one month, or at least one year after completion of the implantation procedure), changes in the magnitude of the variable tension are radiographically monitoring, from outside the patient's body, over time after the implantation of tension system 110. (Completion of the implantation procedure may be measured from the application of the tension between the first and the second target sites 150A and 150B during the implantation procedure.) For example, in FIG. 3E, tension system 110 is shown as still applying some tension between first and second tissue anchors 120A and 120B, but less than initially upon completion of the implantation procedure. Alternatively, tension system 10 may have ceased to apply any tension, such as shown in FIG. 2C for tension system 10. A physician may evaluate the continued efficacy of tension system 110 based on the radiographically-monitored variable tension, without the need to perform an invasive and/or more complex diagnostic procedure, and may decide to perform a follow-up adjustment procedure if necessary.

It is noted that wire 164 serves as a spring for implantable force gauge 130 both during application of tension, as shown in FIGS. 3C-D, and loss of tension, as shown in FIG. 3E. Wire 164 thus serves two functions: (1) as a spring for the force gauge, and (2) as an anchor for anchoring first tissue-coupling element 122A to the cardiac tissue.

Reference is still made to FIGS. 3A-E. For some applications, first tissue anchor 120A is implanted using one or more of the techniques described in PCT Publication WO 2016/087934, such as with reference to FIGS. 14A-D and/or 16, and/or in US Patent Application Publication 2016/0262741, such as with reference to FIGS. 14-AD, 15A-C, and/or 16.

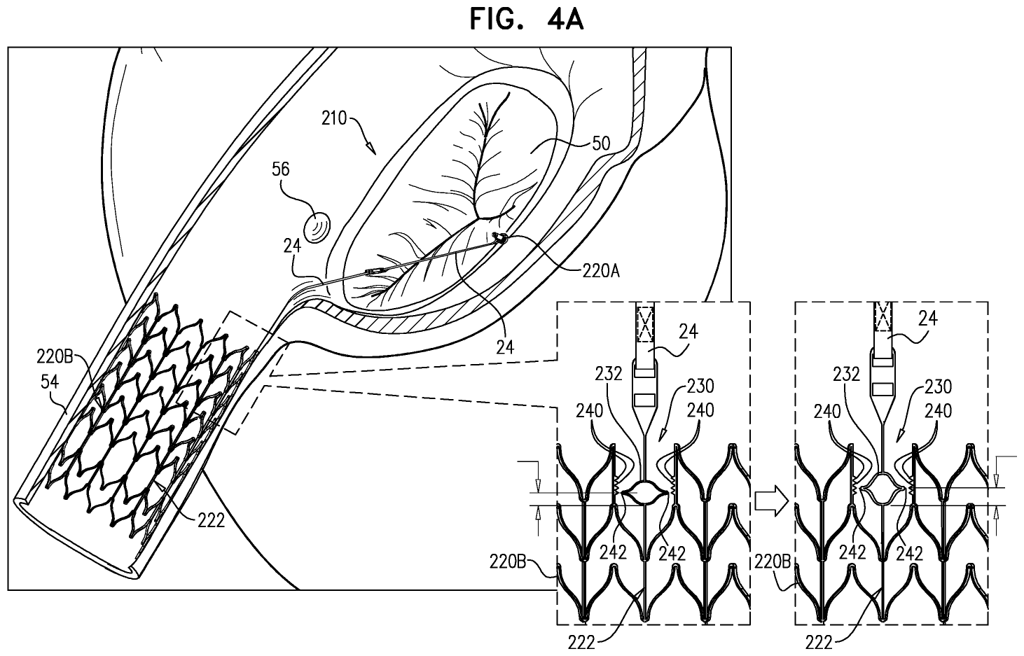

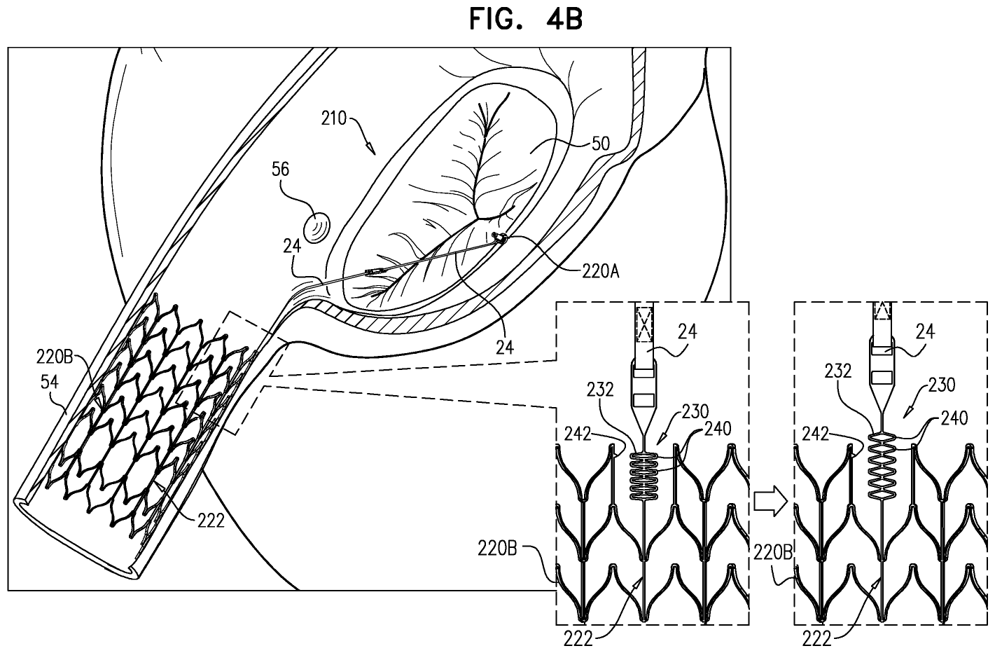

Reference is now made to FIGS. 4A-B, which are schematic illustrations of respective configurations of a tension system 210 for applying variable tension between two target sites in a patient's body, in accordance with respective applications of the present invention. Tension system 210 typically comprises (a) first and second tissue anchors 220A and 220B, which comprise a first tissue-coupling element and a second tissue-coupling element 222, respectively, which are configured to be anchored to the two target sites, respectively, and (b) one or more tethers 24, which are configured to couple together first and second tissue anchors 220A and 220B.

For some applications, as shown in FIGS. 4A-B, second tissue-coupling element 222 comprises a stent that comprises a plurality of struts. Alternatively or additionally, for some applications, the first tissue-coupling element is helical; for other applications, the first tissue-coupling element comprises first tissue-coupling element 122A, described hereinabove with reference to FIGS. 3A-E, or another tissue-coupling element. For some applications, fibrous glue is applied to one or both of the tissue-coupling elements to help secure the anchor in place and minimize detachment. Optionally, tissue-growth-enhancing coating is also applied to one or both of the tissue-coupling elements.

Tension system 210 further comprises an implantable force gauge 230, which is coupled between the one or more tethers 24 and second tissue-coupling element 222. Typically, implantable force gauge 230 is mechanical and does not operate using electricity or any non-mechanical energy.

Implantable force gauge 230 is configured to provide a radiographically-discernible indication of a magnitude of the variable tension between the two target sites, thereby enabling (a) radiographically ascertaining, from outside the patient's body, of the magnitude of the variable tension during implantation of tension system 210, and (b) radiographically monitoring, from outside the patient's body, of changes in the magnitude of the variable tension over time after the implantation of tension system 210.

Implantable force gauge 230 comprises a longitudinally deformable element 232. For some applications, longitudinally deformable element 232 comprises a spring, which is configured to elastically deform upon application of up to a certain force. For other applications, longitudinally deformable element 232 is longitudinally plastically deformable, upon application of beyond a certain force, in which case implantable force gauge 230 measures greatest force ever applied between the between the two target sites.

Implantable force gauge 230 comprises a plurality of radiographically-discernible fiducial markers 240 and a radiographically-discernible pointer 242. Implantable force gauge 230 is arranged such that pointer 242 moves longitudinally with respect to fiducial markers 240 so as to provide the radiographically-discernible indication of the magnitude of the variable tension. Pointer 242 longitudinally coincides with different ones of fiducial markers 240 at different respective values of the magnitude of the variable tension, as shown in the two blow-ups in FIG. 4A and in the two blow-ups in FIG. 4B. Optionally, pointer 242 has a radiopacity different from a radiopacity of fiducial markers 240.

For some applications, as shown in FIG. 4A, fiducial markers 240 are fixed to second tissue-coupling element 222, e.g., one or more struts of a stent, and pointer 242 is fixed to longitudinally deformable element 232. For other applications, as shown in FIG. 4B, pointer 242 is fixed to second tissue-coupling element 222, e.g., one or more struts of a stent, and fiducial markers 240 are fixed to longitudinally deformable element 232.

Implantable force gauge 230 may be implanted and used using techniques described hereinabove with reference to FIGS. 2A-C and/or FIGS. 3A-E, mutatis mutandis.

In an application of the present invention, a force gauge is provided in an external handle of an implant delivery tool, and is used to measure the applied variable tension between two or more tissue anchors during and/or after implantation of the tissue anchors.

As used in the present application, including in the claims, a “pointer” need not have any particular shape; for example, a pointer need not have a long thin shape, such as an arrow.

The scope of the present invention includes embodiments described in the following applications, which are assigned to the assignee of the present application and are incorporated herein by reference. In an embodiment, techniques and apparatus described in one or more of the following applications are combined with techniques and apparatus described herein: U.S. Pat. No. 8,475,525 to Maisano et al.; U.S. Pat. No. 8,961,596 to Maisano et al.; U.S. Pat. No. 8,961,594 to Maisano et al.; PCT Publication WO 2011/089601; U.S. Pat. No. 9,241,702 to Maisano et al.; PCT Publication WO 2013/011502; US Provisional Application 61/750,427, filed Jan. 9, 2013; US Provisional Application 61/783,224, filed Mar. 14, 2013; PCT Publication WO 2013/179295; U.S. Provisional Application 61/897,491, filed Oct. 30, 2013; U.S. Provisional Application 61/897,509, filed October 30, 2013; U.S. Pat. No. 9,307,980 to Gilmore et al.; PCT Publication WO 2014/108903; PCT Publication WO 2014/141239; US Provisional Application 62/014,397, filed Jun. 19, 2014; PCT Publication WO 2015/063580; US Patent Application Publication 2015/0119936; U.S. Provisional Application 62/086,269, filed Dec. 2, 2014; U.S. Provisional Application 62/131,636, filed Mar. 11, 2015; U.S. Provisional Application 62/167,660, filed May 28, 2015; PCT Publication WO 2015/193728; PCT Publication WO 2016/087934; US Patent Application Publication 2016/0242762; PCT Publication WO 2016/189391; US Patent Application Publication 2016/0262741; and U.S. Provisional Application 62/376,685, filed Aug. 18, 2016.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A tension system (10) for applying variable tension between two target sites in a patient's body, the tension system (10) comprising:
first and second tissue anchors (20A, 20B), comprising respective first and second tissue-coupling elements (22A, 22B) that are configured to be anchored to the two target sites, respectively;
first and second tethers (24A, 24B), coupled to the first and the second tissue anchors (20A, 20B), respectively; and
an implantable force gauge 30, configured to provide a radiographically-discernible indication of a magnitude of the variable tension between the two target sites, thereby enabling (a) radiographically ascertaining, from outside the patient's body, of the magnitude of the variable tension during the applying of the variable tension, and (b) radiographically monitoring, from outside the patient's body, of changes in the magnitude of the variable tension over time after implantation of the tension system (10), characterized in that:
the implantable force gauge (30) comprises first and second components (31A, 31B), which are fixed to the first and the second tethers (24A, 24B), respectively, and which are non-integral with each other and are configured to be coupled together in situ so as to couple the first and the second tissue anchors (20A, 20B) together via the first and the second tethers (24A, 24B), for applying the variable tension between the two target sites.

2. The tension system according to claim 1, wherein the first component (31A) of the implantable force gauge (30) comprises a longitudinally deformable element (32).

3. The tension system according to claim 2, wherein the longitudinally deformable element (32) comprises a spring.

4. The tension system according to claim 2, wherein the longitudinally deformable element (32) is longitudinally plastically deformable.

5. The tension system according to any one of claims 1-4,
wherein the implantable force gauge (30) comprises a plurality of radiographically-discernible fiducial markers (40) and a radiographically-discernible pointer (42),
wherein the implantable force gauge (30) is arranged such that the pointer (42) moves longitudinally with respect to the fiducial markers (40) so as to provide the radiographically-discernible indication of the magnitude of the variable tension, and
wherein the pointer (42) longitudinally coincides with different ones of the fiducial markers (40) at different respective values of the magnitude of the variable tension.

6. The tension system according to claim 5, wherein the pointer (42) has a radiopacity different from a radiopacity of the fiducial markers (40).

7. A tension system for applying variable tension between first and second target sites in a patient's body, the tension system comprising:
(a) a first tissue anchor, which comprises:
an anchor shaft;
a first tissue-coupling element, which (i) extends from a distal end of the anchor shaft, and (ii) comprises a wire, which is shaped as an open shape when the first tissue anchor is unconstrained by a deployment tool, and which is configured to be anchored to the first target site; and
a flexible elongate tension member, which includes (i) a distal portion that is fixed to a site on the open shape, (ii) a proximal portion, at least a portion of which runs alongside at least a portion of the anchor shaft, and (iii) a crossing portion, which crosses from the site on the open shape to the distal end of the anchor shaft when the first tissue anchor is unconstrained by the deployment tool, wherein the first tissue anchor is configured to allow relative axial motion between the at least a portion of the anchor shaft and the at least a portion of the proximal portion of the flexible elongate tension member when the first tissue anchor is unconstrained by the deployment tool;
(b) a second tissue anchor, which comprises a second tissue-coupling element that is configured to be anchored to the second target site;
(c) one or more tethers, configured to couple the flexible elongate tension member to the second tissue anchor and apply the variable tension between the first and the second target sites; and
(d) an implantable force gauge, which comprises:
one or more radiographically-discernible fiducial markers, which are disposed on the proximal portion of the flexible elongate tension member; and
a radiographically-discernible pointer, which is disposed on the anchor shaft,
wherein the implantable force gauge is arranged such that the one or more fiducial markers move longitudinally with respect to the pointer so as to provide a radiographically-discernible indication of a magnitude of the variable tension between the first and the second target sites, thereby enabling (a) radiographically ascertaining, from outside the patient's body, of the magnitude of the variable tension during the applying of the variable tension, and (b) radiographically monitoring, from outside the patient's body, of changes in the magnitude of the variable tension over time after implantation of the tension system, wherein the pointer longitudinally coincides with different ones of the fiducial markers at different respective values of the magnitude of the variable tension.

8. A method for applying variable tension between two target sites in a patient's body, the method comprising:
anchoring, to the two target sites, respectively, first and second tissue-coupling elements of respective first and second tissue anchors of a tension system;
coupling the first and the second tissue anchors together in situ via first and second tethers of the tension system, coupled to the first and the second tissue anchors, respectively, by coupling together in situ first and second components of an implantable force gauge of the tension system, wherein the first and the second components are non-integral with each other and are fixed to the first and the second tethers, respectively, and wherein the implantable force gauge is configured to provide a radiographically-discernible indication of a magnitude of the variable tension between the two target sites;
applying the variable tension between the two target sites via the first and the second tethers and the implantable force gauge; and
radiographically ascertaining, from outside the patient's body, the magnitude of the variable tension during the applying of the variable tension, by radiographically observing the radiographically-discernible indication of the magnitude of the variable tension.

9. The method according to claim 8, further comprising radiographically monitoring, from outside the patient's body, changes in the magnitude of the variable tension over time after implantation of the tension system.

10. The method according to claim 8, wherein the first component of the implantable force gauge comprises a longitudinally deformable element.

11. The method according to claim 10, wherein the longitudinally deformable element comprises a spring.

12. The method according to claim 10, wherein the longitudinally deformable element is longitudinally plastically deformable.

13. The method according to claim 8, wherein the implantable force gauge comprises a plurality of radiographically-discernible fiducial markers and a radiographically-discernible pointer, wherein the implantable force gauge is arranged such that the pointer moves longitudinally with respect to the fiducial markers so as to provide the radiographically-discernible indication of the magnitude of the variable tension, and wherein the pointer longitudinally coincides with different ones of the fiducial markers at different respective values of the magnitude of the variable tension.

14. The method according to claim 13, wherein the pointer has a radiopacity different from a radiopacity of the fiducial markers.

15. The method according to claim 8, wherein the two target sites are two cardiac tissue target sites, respectively.

16. A method for applying variable tension between first and second target sites in a patient's body, the method comprising:
anchoring, to the first target site, a wire of a first tissue-coupling element of a first tissue anchor of a tension system, wherein the first tissue anchor further comprises an anchor shaft, wherein the first tissue-coupling element extends from a distal end of the anchor shaft, wherein the wire is shaped as an open shape when the first tissue anchor is unconstrained by a deployment tool, and wherein the first tissue anchor further comprises a flexible elongate tension member, which includes (i) a distal portion that is fixed to a site on the open shape, (ii) a proximal portion, at least a portion of which runs alongside at least a portion of the anchor shaft, and (iii) a crossing portion, which crosses from the site on the open shape to the distal end of the anchor shaft when the first tissue anchor is unconstrained by the deployment tool, wherein the first tissue anchor is configured to allow relative axial motion between the at least a portion of the anchor shaft and the at least a portion of the proximal portion of the flexible elongate tension member when the first tissue anchor is unconstrained by the deployment tool;
anchoring, to the second target site, a second tissue-coupling element of a second tissue anchor of the tension system, such that one or more tethers couple the flexible elongate tension member to the second tissue anchor and apply the variable tension between the first and the second target sites; and
radiographically ascertaining, from outside the patient's body, the magnitude of the variable tension during the applying of the variable tension, by radiographically observing a radiographically-discernible indication of the magnitude of the variable tension provided by an implantable force gauge of the tension system, which comprises (a) one or more radiographically-discernible fiducial markers, which are disposed on the proximal portion of the flexible elongate tension member, and (b) a radiographically-discernible pointer, which is disposed on the anchor shaft, wherein the implantable force gauge is arranged such that the one or more fiducial markers move longitudinally with respect to the pointer so as to provide the radiographically-discernible indication of the magnitude of the variable tension between the first and the second target sites, and the pointer longitudinally coincides with different ones of the fiducial markers at different respective values of the magnitude of the variable tension.

17. The method according to claim 16, further comprising radiographically ascertaining, from outside the patient's body, changes in the magnitude of the variable tension over time after implantation of the tension system.

18. The method according to claim 16, wherein the first and the second target sites are two cardiac tissue target sites, respectively.
